(19) United States
(12) Patent Application Publication
(10) Pub. No.: US 2005/0214350 A1
(43) Pub. Date: Sep. 29, 2005

(54) Antimicrobial sheets for environmental and human protection and prevention

(76) Inventor: Kaila R. Thompson, Naples, FL (US)

Correspondence Address: Kaila R. Thompson, 680 92nd Ave. North, Naples, FL, 34108, US

(21) Appl. No.: 10/815,197

(22) Filed: Mar. 29, 2004

(51) Int. Cl. A61K 9/70

(52) U.S. Cl. 424/443

(57) ABSTRACT

Antimicrobial sheets for prevention, environmental and human protection. Preferably the sheets are formed into linear, square and rectangular sheets from a singular form to a multi-layered configuration that may be exposed to environmental natural elements occasionally and or permanently which may interact with human contact.

### FIELD OF INVENTION

The invention lies in the field of the protection of human life.

### SUMMARY OF THE INVENTION

A monofilament sheet and or multi layered sheet with the antimicrobial Triclosan, and or antimicrobial Microban incorporated therein. The sheet or tape is fully capable of covering all t types of surfaces that may contain harmful bacteria and or viruses that may be transmitted upon human contact. The material and application would prevent the spread of numerous diseases and promotes healthy people from becoming ill.

### DESCRIPTION OF THE DRAWINGS

FIG. 1 A side plan view section of the types of sheets and or tapes in a linear form; covering various widths and thicknesses of the material containing the antimicrobials which will be dispensed in certain sizes that will have a perforated edge, allowing the linear sheet or tape to be removed exposing a re-newed sheet or tape for continued exposure. The cross section demonstrates the material containing the antimicrobial compound.

FIG. 2 A top plan view section of the types of sheets and or tapes in a rectangular form; covering various widths and thicknesses of the material containing the antimicrobials which will be dispensed in certain individual sizes, allowing the rectangular sheet or tape to be removed exposing a re-newed sheet or tape for continued exposure and longevity. The cross section demonstrates the material containing the antimicrobial compound.

FIG. 3 A top plan view cross-section of the types of sheets and or tapes in a square form; covering various widths and thicknesses of the material containing the antimicrobials which will be dispensed in certain sizes that will have a perforated edge, allowing the square sheet or tape to be removed exposing a re-newed sheet or tape for continued exposure. The cross section demonstrates the material containing the antimicrobial compound.

FIG. 4 A side plan view cross section of the types of sheets and or tapes in a square or rectangular form; covering various widths and thicknesses of the material containing the antimicrobials which will be dispensed in certain sizes that will have a perforated edge, allowing the square sheet or tape to be removed exposing a re-newed sheet or tape for continued exposure. The cross section demonstrates the material containing the antimicrobial compound.

FIG. 5 The antimicrobial sheet refillable apparatus that will dispense the antimicrobial linear Sheets and or tape in a configuration for human contact and prolonged exposures, the apparatus is designed to be attached to either to round and or square surfaces of various size, allowing the linear sheet and or tape to be affixed for dispensing, the apparatus allows for re-filling the linear roll for continued dispensing. The cross section (FIG. (6,7 and 8)) describes the mechanical nature of the apparatus along with the demonstration of the material containing the antimicrobial compound.

FIG. 6 A side view section of the apparatus referred to in (FIG. 5) indicating the cross section of the antimicrobial layered sheets which depicts various size configurations that this apparatus will Coantimicrobial-layeredications. The antimicrobial sheets used in accordance with this apparatus can be dispensed easily as an article in which application of the invention is applied that may have human contact, thus by application of invention, either eliminating and or preventing the spread of bacterial and or viral organisms. The scope of the invention lies within the unique nature of the material and its use as applied for human contact barrier. Such invention to be applied in public and or private areas to prevent the spread of harmful diseases.

FIG. 7 A top view section of the apparatus referred to in (FIG. 5) indicating the cross section of the antimicrobial-layereded sheets which depicts various size configurations that this apparatus will conform to various applications. Additional articles as examples in a top and side view of an article in which application of the invention is applied that may have human contact in FIG. (9,10,11 and 12), thus by application of invention, either eliminating and or preventing the Spread of bacterial and or viral organisms. The scope of the invention lies within the unique nature of the material and its use as applied for human contact barrier. Such invention to be applied in public and or private areas to prevent the spread of harmful diseases.

FIG. 8 A cross section view of the adjustable bracket used with the antimicrobial sheets apparatus in FIG. (5) This illustration depicts the manner in which the sheets can be applied to the antimicrobial sheet apparatus and applied to various applications via a snap on clamp.

FIG. 9 Example (1) of handles on a shopping cart in which the invention is applied via the antimicrobial sheet apparatus on the shopping cart handle.

FIG. 10 Example (2) of handle or handle(s) on refrigerators in which the invention is applied via the antimicrobial sheet apparatus on the refrigerator door handles.

FIG. 11 Example (3) of various door entrances and exits in which the antimicrobial invention is applied via a mat configuration in either square and or rectangular mats, these few examples as doors used for a shower, airport, grocery store does not represent the extent of which the invention can be applied to in numerous other various applications. Thus by application of antimicrobial sheet invention, either eliminating and or preventing the spread of bacterial and or Viral organisms. The scope of the invention lies within the unique nature of the material and its use as applied for human contact barrier. Such invention to be applied in public and or private areas to prevent the spread of harmful diseases.

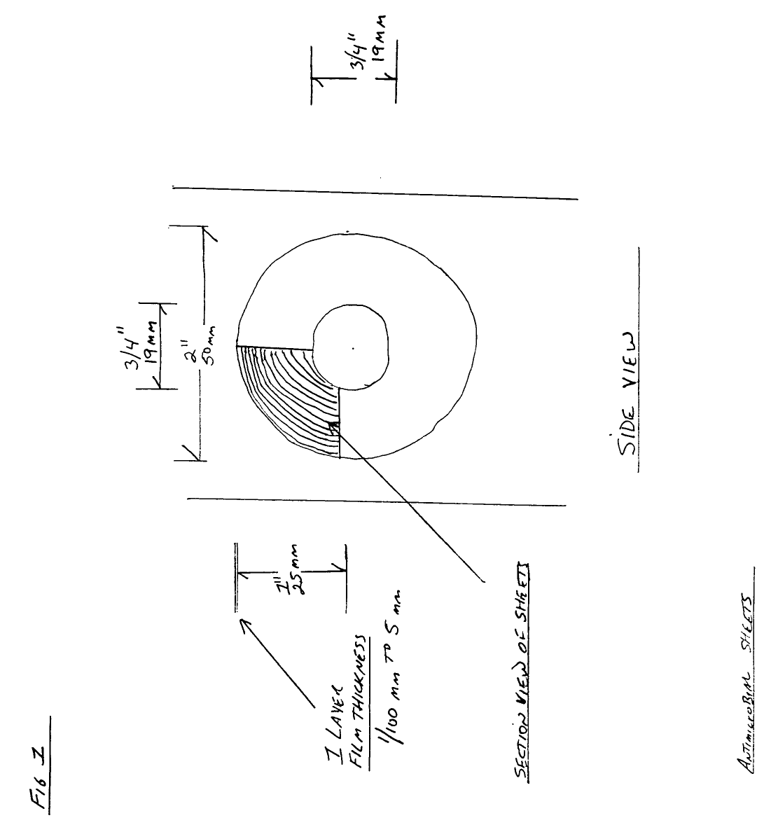

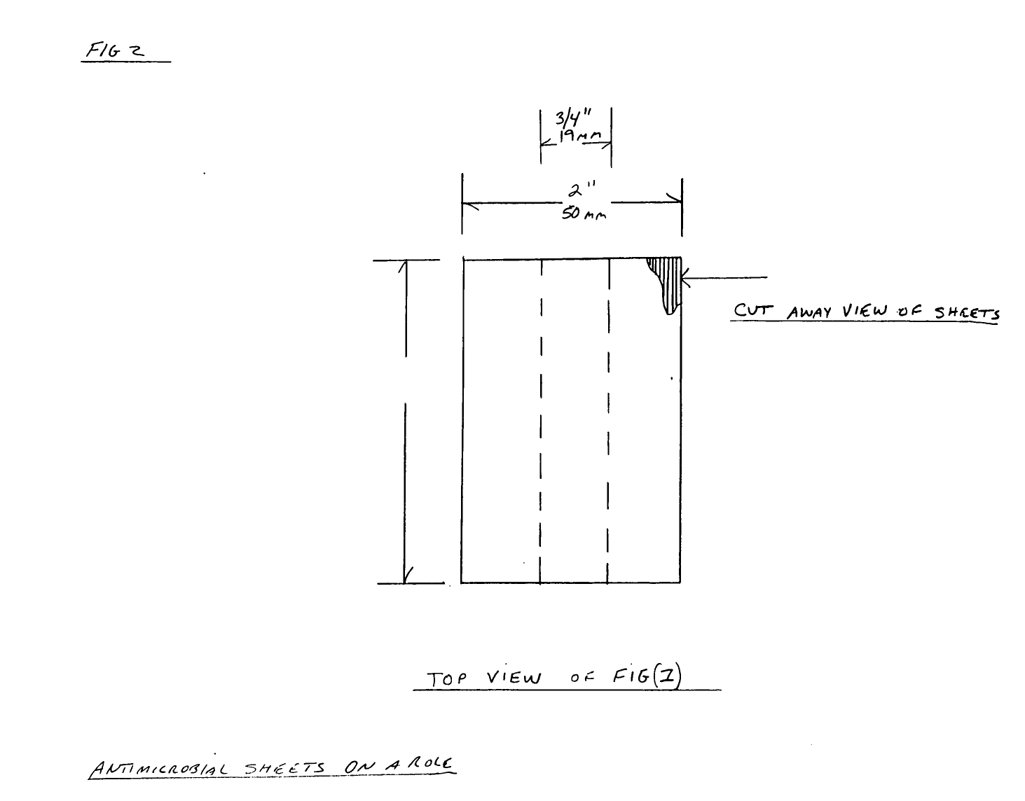

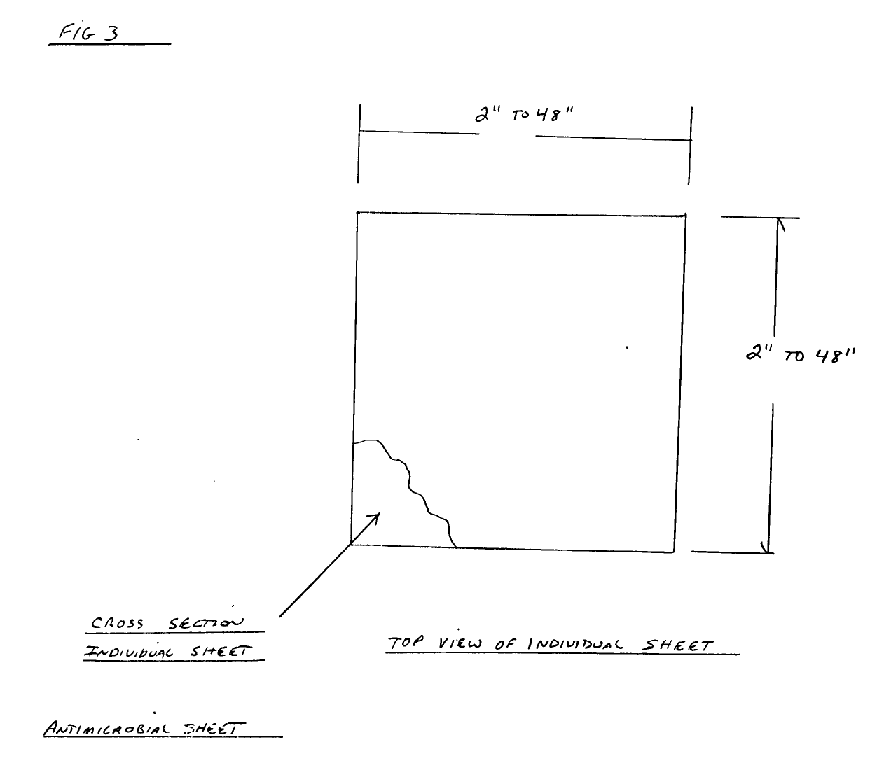

These figures, which are idealized, are not to scale and are intended to be merely illustrative and non limiting.

### DETAILED DESCRIPTION OF THE PREFERRED FORM OF THE INVENTION

Applicant's monofilament and or multi-layered material may contain one or more of several antimicrobial compounds, some of which are presently used in commercially known plastics and soaps. Two of the compounds known to be used in plastics and soaps are triclosan (2,4,4-trichloro-2hydroxy-diphenylether) and n-alkyl dimethylbenzyl ammonium chloride dimethyl benzyl ammonium chloride. The invention relates to the prevention of the spread of infections and diseases.

While I have disclosed in this specification and shown in it's drawing a detailed explanation of my invention, the true scope and extent of my invention is set forth only in the following claims.

1. Antimicrobial material that comes into contact with natural human non invasive touch, would prevent the spread of infectious diseases. The use microorganisms have been known for a long time. The use of anitmicrobial active compounds are constructed in a pure form. Upon the touch and or contact from one human which may carry harmful bacterial, the invention would prevent such as harmful bacteria being spread and thus the active bacteria would be destroyed.

2. The material containing antimicrobial compound would be in a monofilament and or multi-layered sheet form. The form would contain active antimicrobial compounds that upon the release of any harmful diseases would be destroyed, thus preventing the spread of harmful bacteria and Viral infections.

3. The purpose of the invention is to prevent the spread of detrimental diseases that are passed on from one human unto another from touching, stepping and or breathing. The scope of the invention is not contained within any prior art and therefore provides a new concept of prevention of infectious diseases and does not in any manner relate to invasive applications as outlined in claim.

4. The invention shall consist of a single or multi-layered antimicrobial hydrophobic material which will be synthetic or chemically induced, with polymers, such as polyethylene, polypropylene, polyurethane, polyamide, polyester, polyvinyl chloride, polytetrafluororthylene or polymers which are prepared by covalent linking of hydrophilic substances with hydrophobic groups, for example according to EP-B 21 230.

5. The antimicrobial adsorbing properties of hydrophobic materials are known (cf. D. F. Gerson et al., Biochim. Biophys. Acta, 602 (1980, 506-510): Y. Fujioka-Hirai. Et al., J. of Biochemical Materials Research, Volume 21, 913-20 (1987(; S. Hjecrten et al., J. of Chromatography 101 (1974), 281-288: M. Fletcher et al., Appl. And Environmental Microbiology, January 1979, 76-72.

1. The invention relates to prevention of harmful infection from human contact in environmental areas with the use of antimicrobial sheets. Antimicrobial material that comes into contact with natural human non invasive touch or contact, would prevent the spread of infectious diseases. The use microorganisms have been known for a long time. The use of anitmicrobial active compounds are constructed in a pure form. Upon the touch and or contact from one human which may carry harmful bacterial, the invention would prevent such as harmful bacteria being spread and thus the active bacteria would be destroyed and not allowed to be spread to other humans.

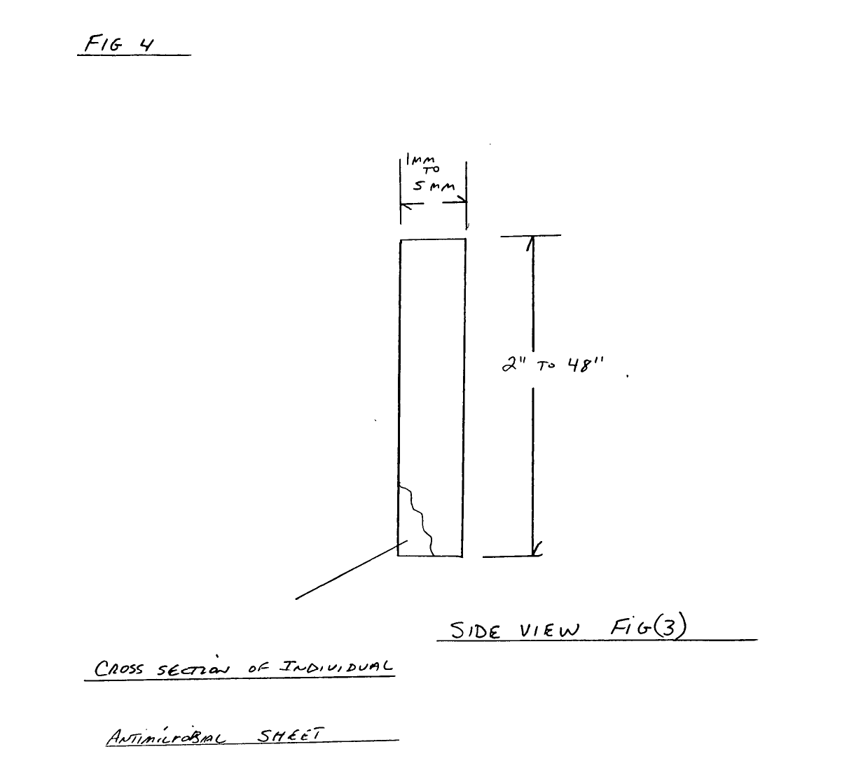

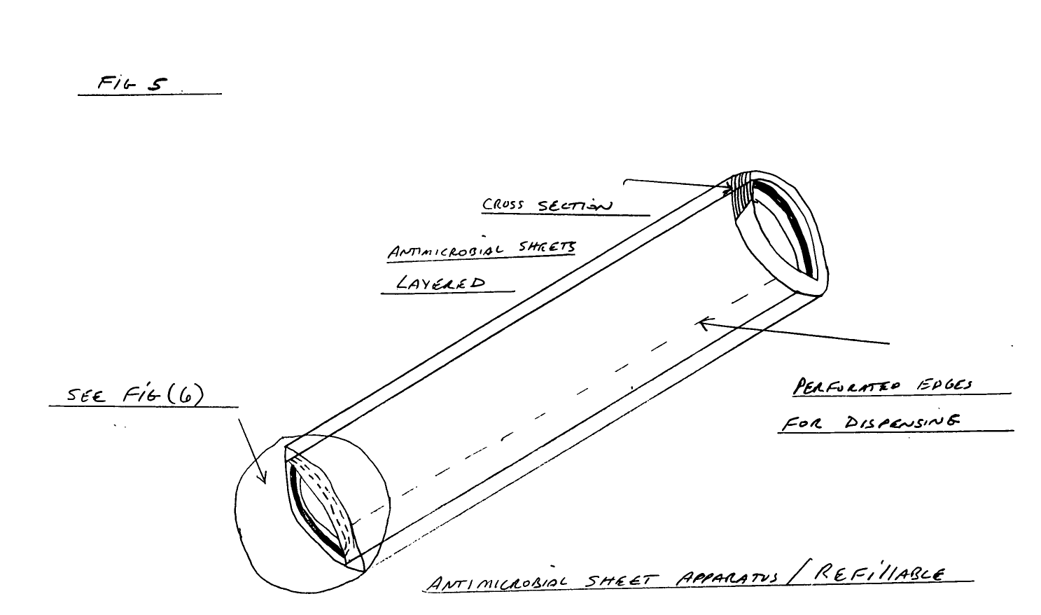

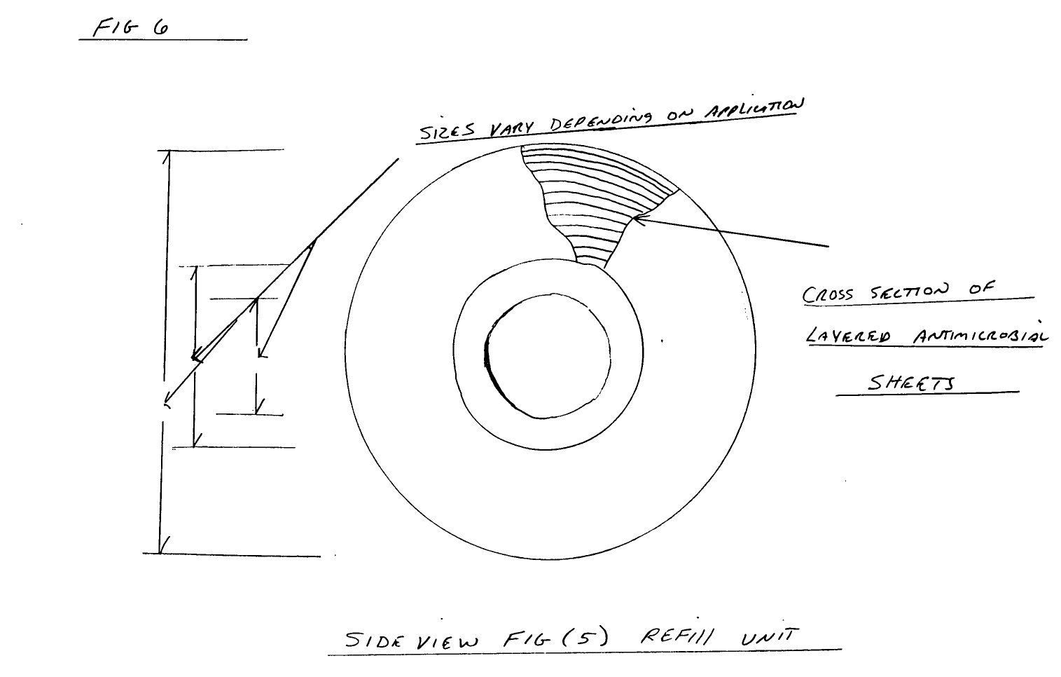

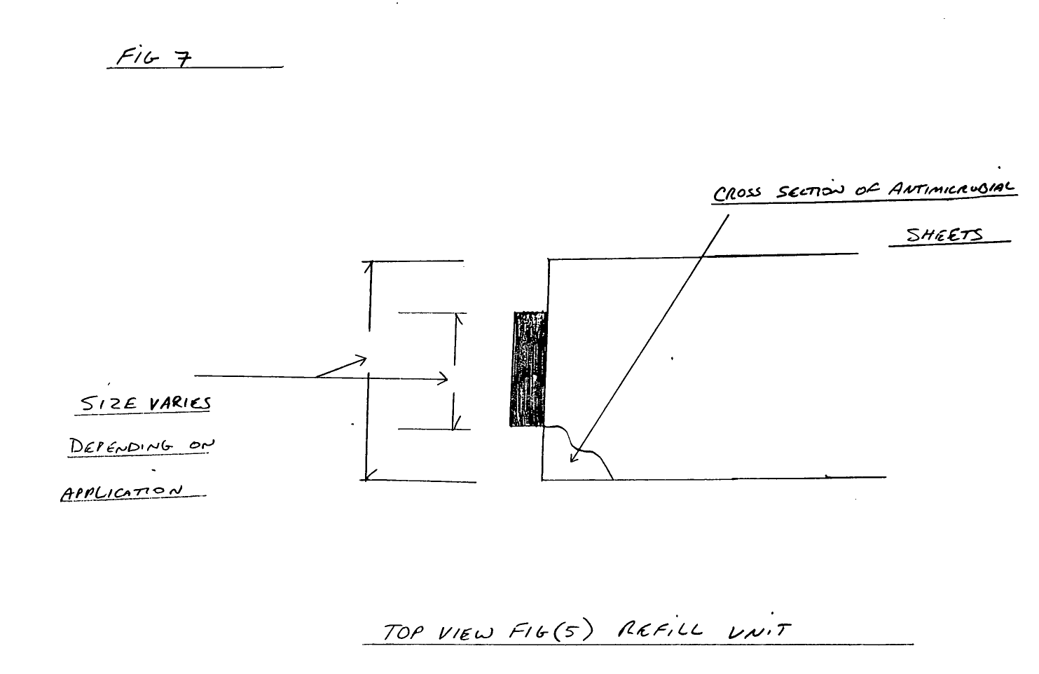

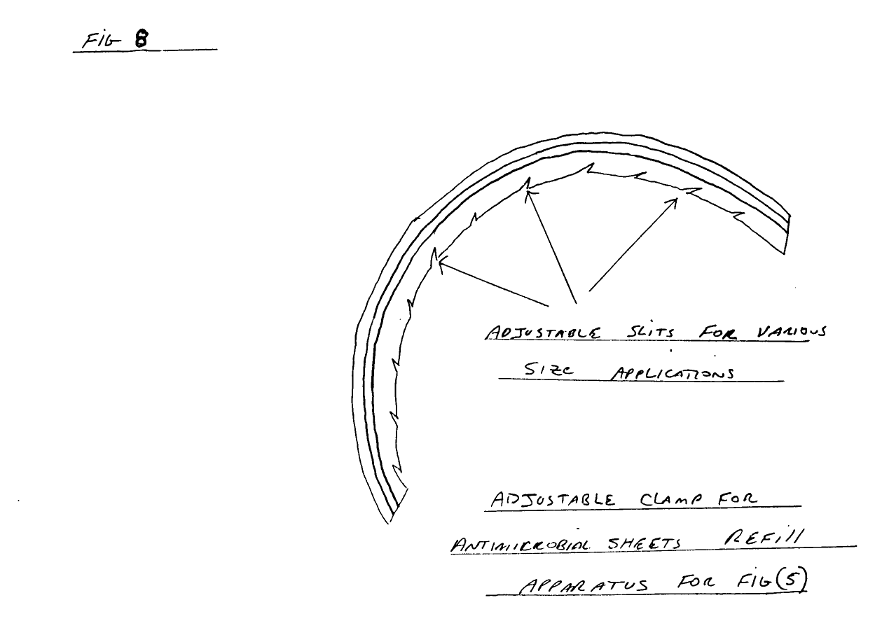

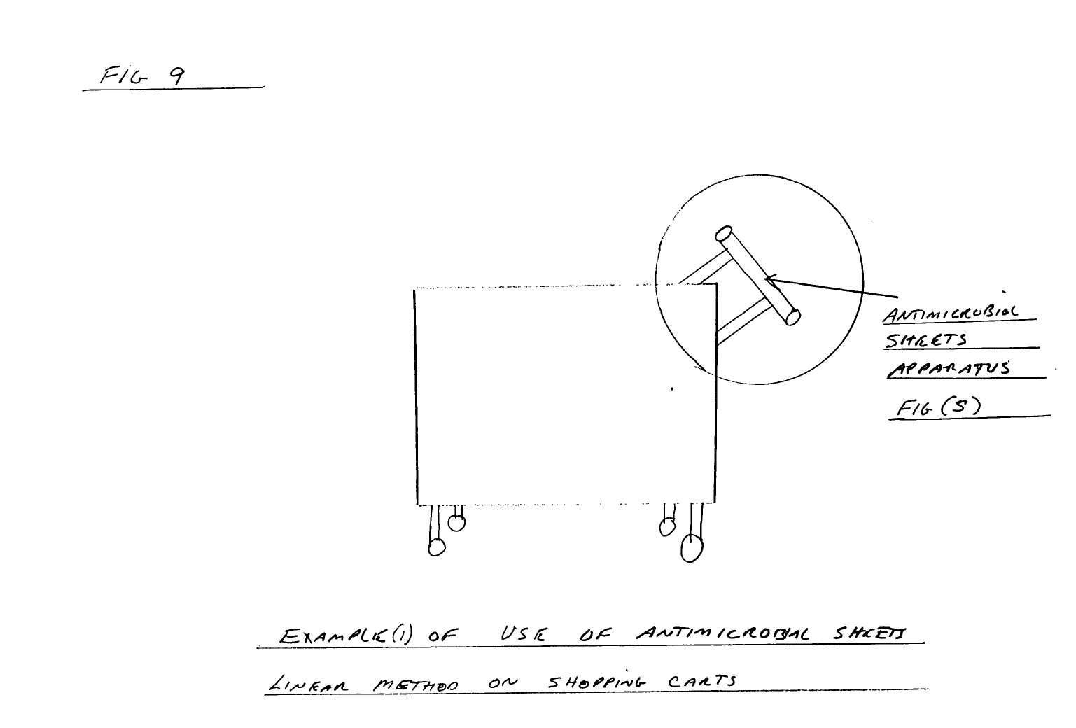

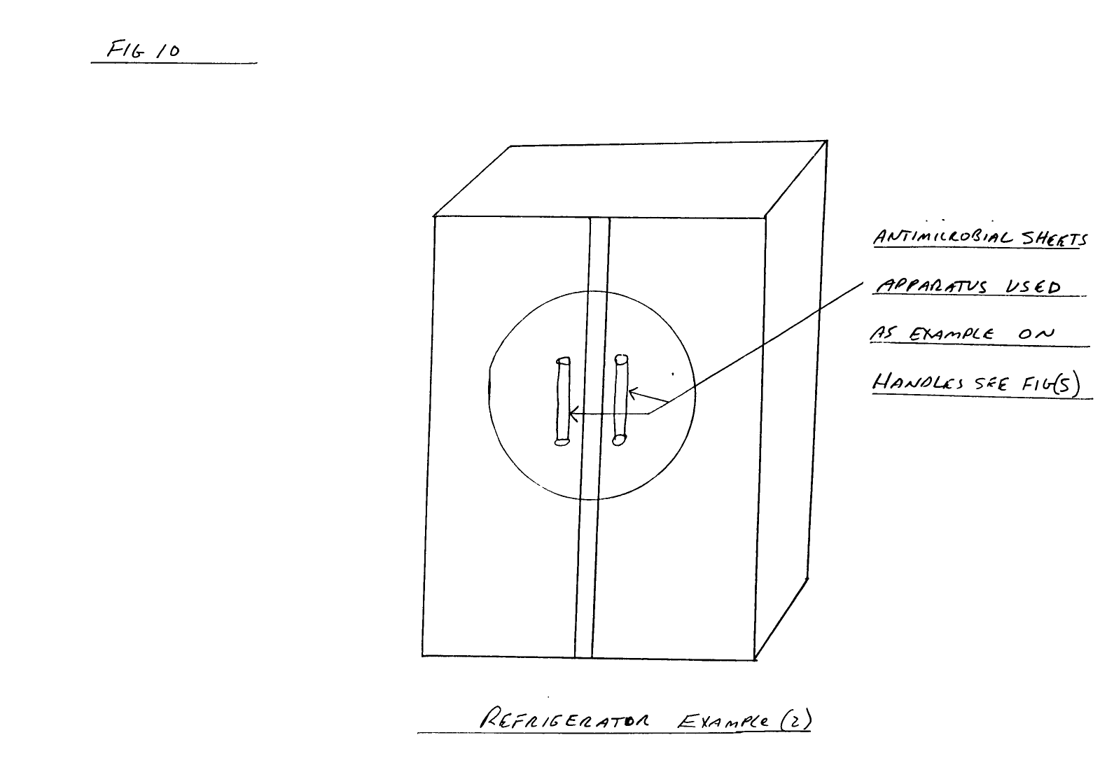

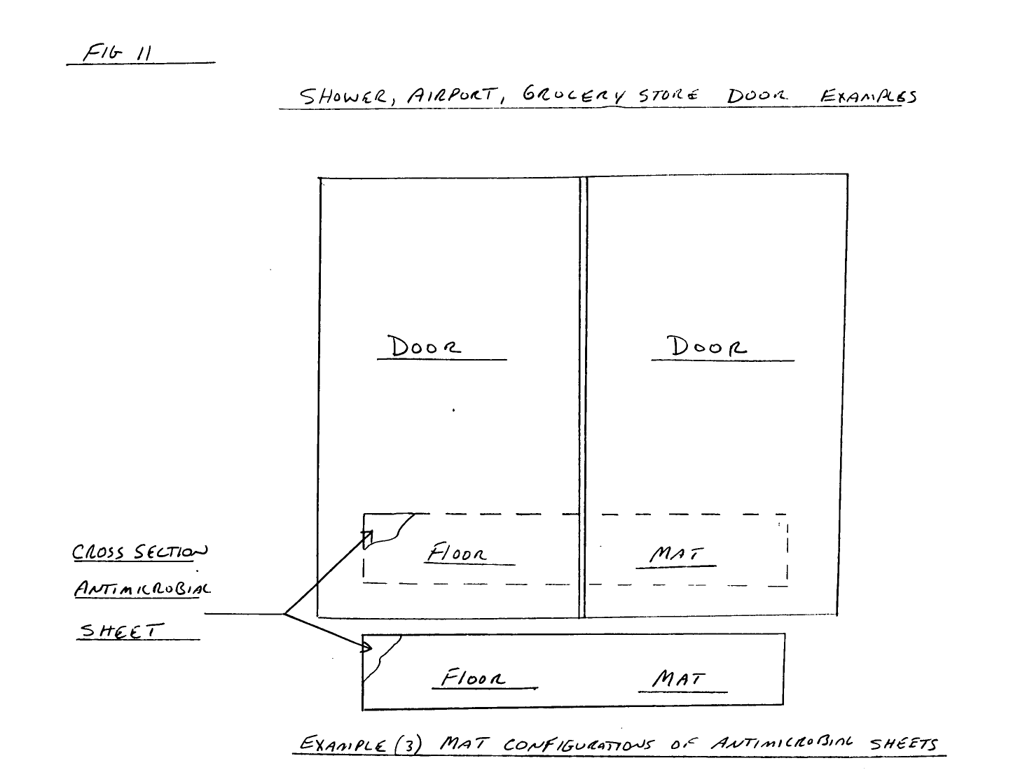

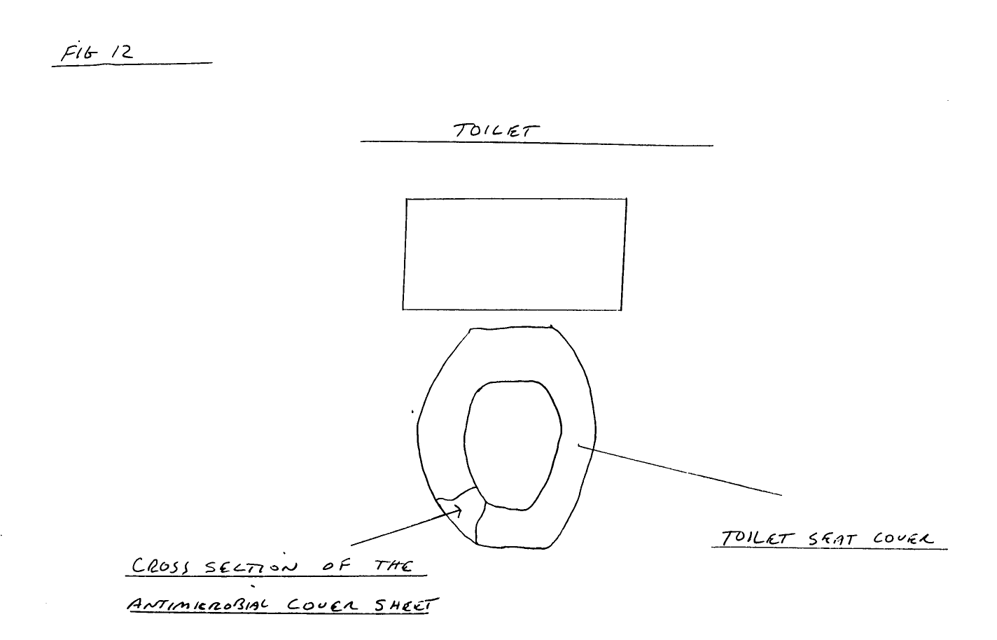

2. An antimicrobial sheet as set forth in claim 1 in which mono or multi layered individual sheets are formed in a configuration to be applied to an Apparatus and or applied individually made to conform in some but not limited areas where human contact is made as described in FIGS. 3-7

3. An antimicrobial sheet as set forth in claim 1 which is formed into a square individual sheet applied in areas of human contact as described in FIG.

4. An antimicrobial sheet as set forth in claim 1 which is formed into a individual rectangular sheet

5. An antimicrobial sheet as set forth in claims 1, 2, 3 and 4 which is in the form of an individual sheet and or multi layered individual sheets which is not limited to commercial and residential uses and is large enough to cover all types of surfaces that may contain harmful bacterial and certain viral Pathogens.

6. The antimicrobial invention shall not be toxic or harmful to the human skin, and or shall not provide airborn protection by exmittance. The present invention can be reused many times over a long period of time and users will be able to rely on it as an easy, compact, rugged, inexpensive, and reliable means for dealing with continued human contact that will prevent the spread of harmful diseases.
